(19) United States
(12) Patent Application Publication
(10) Pub. No.: US 2010/0226872 A1
(43) Pub. Date: Sep. 9, 2010

(54) Stabilized Liquid Preservative Compositions

(76) Inventors: John J. Merianos, Wayne, NJ (US); Susan M. Lindstrom, Ramsey, NJ (US); Paul Garelick, South Plainfield, NJ (US)

Correspondence Address: INTERNATIONAL SPECIALTY PRODUCTS;WILLIAM J. DAVIS, ESQ., 1361 ALPS ROAD, WAYNE, NJ, 07470, US

(21) Appl. No.: 12/376,483

(22) PCT Filed: Aug. 8, 2007
(86) PCT No.: PCT/US07/75467

§ 371 Date: May 12, 2010

Related U.S. Application Data
(60) Provisional application No. 60/836,559, filed on Aug. 9, 2006.

(51) Int. Cl. A01N 37/10; A01N 43/50; A61K 8/18; A61Q 5/02; A61Q 5/00; A61Q 1/10; A61Q 5/12; A01P 1/00

(52) U.S. Cl. 424/70.7; 514/544; 514/390; 510/119; 424/70.1; 8/405

(57) ABSTRACT

The present invention is a directed to a composition and method of solubilizing and stabilizing a liquid preservative composition containing an alkyl paraben ingredient which comprises including polyvinyl pyrrolidone (PVP) in said composition. The compositions are particularly useful in personal care products.

### CROSS-REFERENCE TO RELATED APPLICATIONS

The present application claims the benefit of U.S. Provisional Application Ser. No. 60/836,559, filed on Aug. 9, 2006, the disclosure of which is hereby incorporated by reference in its entirety.

### FIELD OF THE INVENTION

This invention relates to preservative compositions, and more particularly to easy-to-use, broad spectrum, liquid preservative compositions which contain an alkylparaben solubilized and stabilized with polyvinyl pyrrolidone.

### BACKGROUND OF THE INVENTION

Preservative compositions are well known and widely used in personal care products, including skin, hair and eye care products. International Specialty Products markets Germaben® II and Germaben® II-E liquid preservative compositions which are blends of diazolidinyl urea, methyl paraben and propyl paraben. However, it is desired to provide new preservative formulations which are stable and do not require the use of high chain alkyl parabens such as propyl and butyl and higher alkyl parabens.

### SUMMARY OF THE INVENTION

We have discovered that polyvinyl pyrrolidone inhibits the formation of crystals of lower alkyl, e.g., C₁-C₂ alkyl, parabens, thereby providing enhanced solubility and stability of liquid preservative compositions featuring such lower alkyl parabens.

More particularly, in accordance with this invention there is provided a solubilized and stabilized liquid preservative composition which includes an alkyl paraben and polyvinyl pyrrolidone (PVP).

In another aspect, this invention provides a method of solubilizing and stabilizing a liquid preservative composition containing an alkyl paraben which comprises adding polyvinyl pyrrolidone (PVP) to said composition.

This invention further provides personal care products featuring the above-described liquid preservative composition at a predetermined use level.

### DETAILED DESCRIPTION OF THE INVENTION

While the invention is described hereinafter with respect to preferred embodiments thereof, e.g., a liquid preservative composition comprising diazolidinyl urea in propylene glycol solvent, it is contemplated that the invention can also be practiced in connection with any preservative, that is combinable with an alkyl paraben and any solvent suitable for such preservative.

The alkyl paraben can be present in an amount up to 15% by weight of the composition. Preferably the alkyl paraben is methyl paraben.

The PVP can be present in an amount up to 50,000 ppm (5% by weight) of the composition. However, lesser amounts, e.g., up to 2000 ppm (0.2 wt %), of PVP have been found to be effective. In a particularly preferred embodiment, the PVP is present in an amount of 0.05-0.10 wt % (500-1000 ppm). Preferably the PVP is PVP K-30.

In a preferred embodiment, the liquid preservative composition includes a preservative in addition to the alkyl paraben. A preferred preservative is diazolidinyl urea. The compositions of the invention typically contain a solvent known to be suitable for the preservative selected. In the case of diazolidinyl urea, suitable solvents include propylene glycol. As noted above, the use of other preservatives, such as dimethyldimethylhydantoin (DMDH), and other solvents, such as low molecular weight polyethylene glycols, is specifically contemplated. The selection of preservatives, suitable solvents therefor and suitable amounts of such preservatives and solvents is well within the purview of one of ordinary skill in the art.

In a particularly preferred embodiment, the composition of this invention includes, by weight, about 29-32% diazolidinyl urea, about 11-13% methyl paraben, about 52-60% propylene glycol and about 500-1000 ppm PVP.

The invention further includes method of solubilizing and stabilizing a liquid preservative composition containing an alkyl paraben ingredient by adding polyvinyl pyrrolidone (PVP) to the composition. Preferably, the method comprises adding up to 50,000 ppm PVP to solubilize and stabilize up to 15% by wt of the alkyl paraben.

In a preferred embodiment of the invention, there is provided a solubilized and stabilized liquid preservative composition comprising a preservative ingredient, an alkyl paraben, propylene glycol and polyvinyl pyrrolidone, wherein the PVP functions to solubilize and stabilize said alkyl paraben. In a particularly valuable embodiment of the invention, about 500-1000 ppm PVP K-30 solubilizers and stabilizes about 11-13% methyl paraben, in a composition which includes about 29-32% of diazolidinyl urea preservative and about 52-60% of propylene glycol.

In a preferred method of the invention about 500-1000 ppm PVP K-30 solubilizers and stabilizes about 11-13% methyl paraben, in a composition which includes about 29-32% of diazolidinyl urea preservative and about 52-60% of propylene glycol.

A preferred composition of the invention is shown in Table 1 below:

TABLE 1

| | Wt % |
|---|---|
| Germall ® II (Diazolidinyl Urea) | 29-32% |
| Methylparaben | 11-13% |
| Propylene Glycol | 52-60% |
| PVP K-30 | 500-1000 ppm |

This preservative composition is particularly effective against gram-positive and gram-negative bacteria, yeast and mold making it a useful addition to many personal care formulations. Personal care products including the compositions of this invention suitably contain the composition present at a use level of 0.01-5%, preferably 0.2-3% and most preferably 0.5-1% by weight of the finished product. These personal care products preferably are of pH 2.5-8, more preferably 3.0-7.5. The preservative compositions of this invention are compatible with a broad range of raw materials and are particularly useful in cream, lotion, shampoo, and conditioner applications. The compositions described herein are also useful in a variety of products for personal care, including, but not limited to, gels, glazes, glues, mousses, sprays, fixatives, 2n1 shampoos, temporary hair dyes, semi-permanent hair dyes, permanent hair dyes, straighteners, permanent waves, relaxers, putties, waxes, pomades, moisturizers, mascaras, lip balms and foam enhancers.

The preservative composition is especially useful for personal care formulators who want to move away from formulations containing higher chain alkyl parabens such as propyl paraben and higher alkyl parabens. In a particularly preferred embodiment, the composition of the invention comprises a sole alkyl paraben such as methyl paraben. Such compositions enable formulators to eliminate higher alkyl parabens which sometimes is desirable.

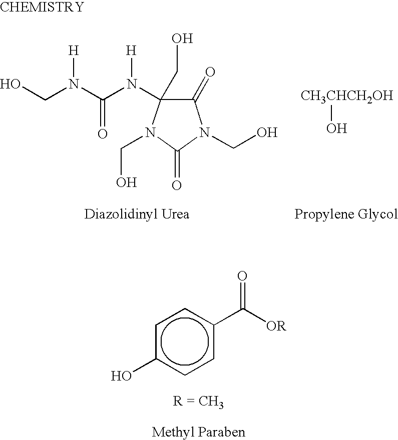

The benefits achieved herein include an easy to use liquid form, broad-spectrum preservation composition, which is temperature and pH stable, and effective within a broad pH range of 2.5-8. Moreover, the compositions described herein are compatible with most cosmetic ingredients, and have a good safety and toxicological profile.

### Preservative System Efficacy in Formulations

A challenge test is a 28-day test used to verify the effectiveness of a preservative system in a personal care formulation. Accordingly, selected personal care formulations were inoculated with microorganisms at the onset of testing (0 hours), then sampled at 48 hours, 7 days, 14 days, 21 days, and 28 days. After 21 days the formulations were re-inoculated with the same microorganisms. Pass/fail criteria was based on a modified Cosmetic Toiletry and Fragrance Association (CTFA) protocol.

The invention composition was tested in several systems and found to effectively preserve a variety of personal care formulations against the microorganisms listed in Table 2

TABLE 2

| Abbreviation | Microorganism |
|---|---|
| SA | Staphylococcus aureus |
| ECOLI | Escherichia coli |
| PSA | Pseudomonas aeruginosa |
| BC | Burkholderia cepacia |
| AN | Aspergillus niger |

The preservative composition of the invention can be conveniently incorporated into personal care products by techniques known in the art. For example, the compositions can be incorporated into hot or cold processed shampoos and into creams and lotions at any stage of the manufacturing process.

A preferred method of addition of the composition herein is to add it slowly to the personal care formulation to be preserved at about 60° C. with good stirring prior to the addition of a fragrance, if any.

An exemplary prototype use formulation in a non-ionic emulsion is shown in Table 3 below.

TABLE 3

| | | W/W | |
|---|---|---|---|
| PHASE | INGREDIENT | % | SUPPLIER |
| A | Deionized Water | 70.10 | |
| | PVM/MA Decadiene | 0.20 | ISP |
| | Crosspolymer (Stabileze ® QM) | | |
| B | Ethylhexyl Palmitate | 10.00 | ISP |
| | (Ceraphyl ® 368) | | |
| | Cetearyl Alcohol and Ceteareth-20 | 2.00 | Amerchol |
| | (Promulgen D) | | |
| | Glyceryl Stearate and | 2.50 | ISP |
| | Laureth 23 (Cerasynt ® 945) | | |
| | Isocetyl Stearate (Ceraphyl ® 494) | 10.00 | ISP |
| C | Triethanolamine (99%) | 0.20 | |
| | Diazolidinyl Urea and Methylparaben | 0.50 | ISP |
| | and Propylene Glycol | | |
| | (Germaben ® M) | | |
| | Polyvinyl pyrrolidone K-30 | 500 ppm | ISP |
| | Deionized Water | qs | |

### Procedure:

1. Sprinkle Stabileze® into water with stirring at RT. Heat phase A to 75° C.
2. Combine phase B, heat to 85° C.
3. Combine phase B and A with stirring, at 75° C.
4: When batch is uniform add phase C.
5. When uniform, turn off heat.
6. Continue stirring thru cool down.
7. Make up water loss and stir at RT.
8. Store in refrigerator.

TABLE 4
Inoculum Concentration (CFU/G)

| | INOCULUM | REINOCULATION |
|---|---|---|
| Organism | (0 Hours) | (21 Days) |
| SA ATCC 6538 | 2.1 × 10⁶ | 1.4 × 10⁶ |
| ECOLI ATCC 8739 | 5.9 × 10⁶ | 7.4 × 10⁶ |
| PSA ATCC 9027 | 4.9 × 10⁶ | 8.1 × 10⁶ |
| BC ATCC 25416 | 2.4 × 10⁶ | 7.8 × 10⁶ |
| AN ATCC 16404 | 1.4 × 10⁶ | 3.3 × 10⁵ |

TABLE 5
Preservative Efficacy Test Results (CFU/G)

| Organism* | 48 Hrs | 7 Days | 14 Days | 21 Days | 28 Days |
|---|---|---|---|---|---|
| SA ATCC 6538 | 1.1 × 10⁶ | <10 | <10 | <10 | <10 |
| ECOLI ATCC 8739 | 4.0 × 10⁶ | <10 | <10 | <10 | <10 |
| PSA ATCC 9027 | <10 | <10 | <10 | <10 | <10 |
| BC ATCC 25416 | <10 | <10 | <10 | <10 | <10 |
| AN ATCC 16404 | <10 | <10 | <10 | <10 | <10 |

*The results above show the efficacy of the invention composition against a broad range of bacteria.

The invention has been described in detail with particular reference to preferred embodiments thereof, but it will be understood that variations and modifications can be, effected within the spirit and scope of the invention.

What is claimed:

1. A method of solubilizing and stabilizing a liquid preservative composition containing an alkyl paraben which comprises adding polyvinyl pyrrolidone (PVP) to said composition.

2. The method of claim 1 which comprises adding PVP to solubilize and stabilize up to 15% by wt of said alkyl paraben.

3. The method of claim 2 wherein said alkyl paraben is C₁-C₂ alkyl paraben.

4. The method of claim 3 wherein said alkyl paraben is methyl paraben.

5. The method of claim 1 wherein said PVP is PVP K-30.

6. The method of claim 1 wherein said composition includes a preservative in addition to said alkyl paraben and a solvent for said preservative.

7. The method of claim 6 wherein said preservative is diazolidinyl urea.

8. The method of claim 6 wherein said solvent is propylene glycol.

9. A solubilized and stabilized liquid preservative composition comprising an alkyl paraben and polyvinyl pyrrolidone, (PVP), wherein said PVP solubilizes and stabilizes said alkyl paraben.

10. The composition of claim 9 wherein said alkyl paraben is C₁-C₂ alkyl paraben.

11. The composition of claim 9 wherein said alkyl paraben is methyl paraben.

12. The composition of claim 9 wherein said PVP is PVP K-30.

13. The composition of claim 9 further including a preservative and a solvent for said preservative.

14. The composition of claim 13 wherein said preservative is diazolidinyl urea.

15. The composition of claim 14 wherein said solvent is propylene glycol.

16. The composition of claim 9 wherein up to 50,000 ppm PVP and up to 15% by weight of said alkyl paraben is present in said composition.

17. The composition of claim 9 which comprises by weight, about 29-32% diazolidinyl area, about 11-13% methyl paraben, about 52-60% propylene glycol and about 500-1000 ppm PVP.

18. A personal care product including the composition of claim 9.

19. The personal care product of claim 15 having a pH of 2.5-8 and wherein said composition is present at a use level of 0.01-5%.
